SEQUENCE LISTING (30 sequences)

SEQ ID NO: 1 - DNA, 31 bp, Unknown
  Produced by Invitrogen Corporation in Carlsbad, CA.  The probes were synthesized on the 200NM scale, with no 3′ or 5′ modifications, purity is standard desalted.
ggccgactcactgcgcgtcttctgtcccgcc

SEQ ID NO: 2 - DNA, 31 bp, Unknown
  Produced by Invitrogen Corporation, Carlsbad, CA.  The probes were synthesized on the 200NM scale with no 3′ or 5′ modifications, purity is standard desalted.
ccggctgagtgacgcgcagaagacagggacg

SEQ ID NO: 3 - DNA, 51 bp, Unknown
  Produced by Invitrogen Corporation in Carlsbad, CA.  The probes were synthesized on the 200NM scale, with no 3′ or 5′ modifications, purity is standard desalted.
ggccgactcactgcgcgtcttctgtcccgccatcacaagtactgggagagg

SEQ ID NO: 4 - DNA, 51 bp, Unknown
  Produced by Invitrogen Corporation in Carlsbad, CA.  The probes were synthesized on the 200NM scale, with no 3′ or 5′ modifications, purity is standard desalted.
ggccgactcactgcgcgtcttctgtcccgccgtctcagaggttaactcacc

SEQ ID NO: 5 - DNA, 51 bp, Unknown
  Produced by Invitrogen Corporation in Carlsbad, CA.   The probeswere synthesized on the 200NM scale, with no 3′ or 5′ modifications, purity is standard desalted.
ggccgactcactgcgcgtcttctgtcccgccttcttatcttcagccccacc

SEQ ID NO: 6 - DNA, 51 bp, Unknown
  Produced by Invitrogen Corporation in Carlsbad, CA.   The probes were synthesized on the 200NM scale, with no 3′ or 5′ modifications, purity is standard desalted.
ggccgactcactgcgcgtcttctgtcccgccataacacggtgtgcaccacg

SEQ ID NO: 7 - DNA, 51 bp, Unknown
  Produced by Invitrogen Corporation in Carlsbad, CA.  The probes were synthesized on the 200NM scale, with no 3′ or 5′ modifications, purity is standard desalted.
ggccgactcactgcgcgtcttctgtcccgcctcccttcctgttgactacag

SEQ ID NO: 8 - DNA, 51 bp, Unknown
  Produced by Invitrogen Corporation in Carlsbad, CA.  The probes were synthesized on the 200NM scale, with no 3′ or 5′ modifications, purity is standard desalted.
ggccgactcactgcgcgtcttctgtcccgcctacccacacgggcttaaaac

SEQ ID NO: 9 - DNA, 51 bp, Unknown
  Produced by Invitrogen Corporation in Carlsbad, CA.  The probes were synthesized on the 200NM scale, with no 3′ or 5′ modifications, purity is standard desalted.
ggccgactcactgcgcgtcttctgtcccgcccactgccagtgtgttttcac

SEQ ID NO: 10 - DNA, 57 bp, Unknown
  Produced by Invitrogen Corp in Carlsbad, CA.  The probes were synthesized on the 200NM scale, with no 3′ or 5′ modifications, purity is standard desalted.
ggccgactcactgcgcgtcttctgtcccgccctgtgacgtatgatggattcaataca

SEQ ID NO: 11 - DNA, 57 bp, Unknown
  Produced by Invitrogen Corp. in Carlsbad, CA. The probes were synthesized on the 200NM scale, with no 3′ or 5′ modifications, purity is standard desalted.
ggccgactcactgcgcgtcttctgtcccgcctcggccatagagctccatcagctgga

SEQ ID NO: 12 - DNA, 56 bp, Unknown
  Produced by Invitrogen Corporation in Carlsbad, CA.  The probes were synthesized on the 200NM scale, with no 3′ or 5′ modifications, purity is standard desalted.
ggccgactcactgcgcgtcttctgtcccgccctgtatggataggaagggatgatgc

SEQ ID NO: 13 - DNA, 60 bp, Unknown
  Produced by Invitrogen Corporation in Carlsbad, CA.  The probes were synthesized on the 200NM scale, with no 3′ or 5′ modifications, purity is standard desalted.
ggccgactcactgcgcgtcttctgtcccgccggctcaggccattcttcattctcgggcct

SEQ ID NO: 14 - DNA, 31 bp, Unknown
  Produced by Invitrogen Corporation in Carlsbad, CA.  The probes were synthesized on the 200NM scale, with no 3′ or 5′ modifications, purity is standard desalted.
ggccgactcactgcgcgtcttctgtcccacc

SEQ ID NO: 15 - DNA, 9 bp, Unknown
  Produced by Invitrogen Corporation in Carlsbad, CA.  The probes were synthesized on the 200NM scale, with no 3′ or 5′ modifications, purity is standard desalted
ctcagccgg

SEQ ID NO: 16 - DNA, 22 bp, Unknown
  Produced by Invitrogen Corporation in Carlsbad, CA.  The probes were synthesized on the 200NM scale, with no 3′ or 5′ modifications, purity is standard desalted.
actgcgcgtcttctgtcccacc

SEQ ID NO: 17 - DNA, 31 bp, Unknown; identical to SEQ ID NO: 14
  Produced by Invitrogen Corporation in Carlsbad, CA.  The probes were synthesized on the 200NM scale, with no 3′ or 5′ modifications, purity is standard desalted.

SEQ ID NO: 18 - DNA, 31 bp, Unknown
  Produced by Invitrogen Corporation in Carlsbad, CA.  The probes were synthesized on the 200NM scale, with no 3′ or 5′ modifications, purity is standard desalted.
ccaccctgtcttctgcgcgtcactcagccgg

SEQ ID NO: 19 - DNA, 31 bp, Unknown; identical to SEQ ID NO: 1
  Produced by Invitrogen Corporation in Carlsbad, CA.  The probes were synthesized on the 200NM scale, with no 3′ or 5′ modifications, purity is standard desalted.

SEQ ID NO: 20 - DNA, 22 bp, Unknown
  Produced by Invitrogen Corporation in Carlsbad, CA.  The probes were synthesized on the 200NM scale, with no 3′ or 5′ modifications, purity is standard desalted.
cttctgcgcgtcactcagccgg

SEQ ID NO: 21 - DNA, 9 bp, Unknown
  Produced by Invitrogen Corporation in Carlsbad, CA.  The probes were synthesized on the 200NM scale, with no 3′ or 5′ modifications, purity is standard desalted.
tgtcccgcc

SEQ ID NO: 22 - DNA, 31 bp, Unknown; identical to SEQ ID NO: 1
  Produced by Invitrogen Corporation in Carlsbad, CA.  The probes were synthesized on the 200NM scale, with no 3′ or 5′ modifications, purity is standard desalted.

SEQ ID NO: 23 - DNA, 31 bp, Unknown
  Produced by Invitrogen Corporation in Carlsbad, CA.  The probes were synthesized on the 200NM scale, with no 3′ or 5′ modifications, purity is standard desalted.
ccgccctgtcttctgcgcgtcactcagccgg

SEQ ID NO: 24 - DNA, 31 bp, Unknown; identical to SEQ ID NO: 1
  Produced by Invitrogen Corporation in Carlsbad, CA.  The probes were synthesized on the 200NM scale, with no 3′ or 5′ modifications, purity is standard desalted.

SEQ ID NO: 25 - DNA, 22 bp, Unknown
  Produced by Invitrogen Corporation in Carlsbad, CA. The probes were synthesized on the 200NM scale, with no 3′ or 5′ modifications, purity is standard desalted.
cttctgcgcgtcactcagccgg

SEQ ID NO: 26 - DNA, 9 bp, Unknown
  Produced by Invitrogen Corporation in Carlsbad, CA.  The probes were synthesized on the 200NM scale, with no 3′ or 5′ modifications, purity is standard desalted.
tgtcccgcc

SEQ ID NO: 27 - DNA, 31 bp, Unknown; identical to SEQ ID NO: 1
  produced by Invitrogen Corporation in Carlsbad, CA.  The probes were synthesized on the 200NM scale, with no 3′ or 5′ modifications, purity is standard desalted.

SEQ ID NO: 28 - DNA, 31 bp, Unknown; identical to SEQ ID NO: 23
  Produced by Invitrogen Corporation in Carlsbad, CA.  The probes were synthesized on the 200NM scale, with no 3′ or 5′ modifications, purity is standard desalted.

SEQ ID NO: 29 - DNA, 31 bp, Unknown; identical to SEQ ID NO: 14
  Produced by Invitrogen Corporation in Carlsbad, CA.  The probes were synthesized on the 200NM scale, with no 3′ or 5′ modifications, purity is standard desalted

SEQ ID NO: 30 - DNA, 47 bp, Unknown
  Produced by Invitrogen Corporation in Carlsbad, CA. The probes were synthesized on the 200NM scale,  with no 3′ or 5′ modifications, purity is standard desalted.
ggccgactcactgcgcgtcttctgtcccgccgaagaccaccggcaag

(19) United States
(12) Patent Application Publication
(10) Pub. No.: US 2003/0207289 A1
(43) Pub. Date: Nov. 6, 2003

(54) Detection of genetic sequences using a bipartite probe

(76) Inventors: Timothy A. Hodge, Cordova, TN (US); Shawn O'Malley, Milleville, MA (US)

Correspondence Address: BUTLER, SNOW, O'MARA, STEVENS & CANNADA PLLC, 6075 POPLAR AVENUE, SUITE 500, MEMPHIS, TN, 38119, US

(21) Appl. No.: 10/233,804

(22) Filed: Sep. 3, 2002

Related U.S. Application Data
(63) Continuation-in-part of application No. 09/945,952, filed on Sep. 4, 2001.

(51) Int. Cl. C12Q 1/68; C12P 19/34

(52) U.S. Cl. 435/6; 435/91.2

(57) ABSTRACT

The present invention concerns a method to improve detection or quantification of a genetic sequence in a genetic sample using a bipartite probe. A bipartite probe is made of a nucleic acid binding sequence capable of hybridizing a target genetic sequence and a binding probe sequence capable of hybridizing to a detectable amplification molecule through a nucleic acid sequence capable of hybridizing to the binding probe sequence of the bipartite probe. The amplification molecule 6 can be a dendrimer that includes a label in its core and/or on any of its arms. Moreover, a secondary signal generation molecule 11 may also be added to the mixture to further increase signal. Similarly, tertiary 20 and quaternary 30 etc. amplification molecules may successively be added to yield increase signal.

### CROSS-REFERENCED TO RELATED APPLICATIONS

This application is a continuation-in-part of U.S. patent application Ser. No. 09/945,952 filed Sep. 4, 2001. The entire disclosure of which is hereby incorporated by reference.

### BACKGROUND OF THE INVENTION

1. Field of the Invention

This invention relates to a method of detection or quantitation of genetic sequences in a sample of DNA using a bipartite probe and more specifically using additional amplification molecules to enhance the detectable signal.

2. Description of the Related Art

Different techniques may be employed in order to label nucleic acid probes. Both direct labeling techniques and indirect labeling have been traditionally used to detect target genetic sequences. Traditionally, indirect labeling techniques use a probe genetic sequence that binds to the selected target genetic sequence that has been modified to contain a specified epitope for an antibody—antigen reaction. The indirect methodology is described in U.S. Pat. Nos. 5,731,158; 5,583,001; 5,196,306 and 5,182,203 (hereby specifically incorporated by reference). In the direct labeling technique the labeled probe hybridizes to a target genetic sequence and is detectable. The probe will be directly modified to contain at least one fluorescent, radioactive or staining molecule per probe, such as cyanine dye, Alexa dye, horseradish peroxidase (HRP) or any other fluorescent signal generation reagent. The fluorescent signal generation reagent includes, for example, Cyanine, Alexa, Texas Red, FITC, DTAF and FAM. FAM is a fluorescein bioconjugate made of carboxyfluorescein succinimidyl ester (e.g. 5-FAM (Molecular Probes, Eugene, Oreg.). DTAF is a fluorescein dichlorotriazine bioconjugate.

The applications for this invention mirror that of PCR and extend further. Any application that requires any detection of specific genetic elements could benefit from this invention. Areas where PCR is not acceptable because of enzymatic difficulties (extension difficulties) dNTP quality, thermocycling nuances, stringency problems would certainly benefit. Traditionally, Southern Blots are used as an alternative assay when PCR proves to be unsuitable.

The most important feature that differentiates this detection technology from PCR is that there is no physical reproduction of the genetic material of interest. The new methodology detects the presence genetic material from the original source DNA.

### BRIEF SUMMARY OF THE INVENTION

The present invention is a process which indirectly detects or quantitates a target genetic sequence in any genetic sample using a bipartite probe. A bipartite probe includes a probe genetic sequence capable of hybridizing with the target genetic sequence and an additional nucleic acid binding probe sequence beyond the probe genetic sequence. Bipartite probes are hybridized with both the target genetic sequence in the DNA sample and amplification probe molecules. Moreover, secondary signal generation molecules may also be added to the mixture to further increase signal. Additional, tertiary and quaternary etc. amplification molecules may successively be added to yield increase signal.

This invention provides a method for detection or quantitation of a target genetic sequence in a genetic sample an assay which involves using a bipartite probe and at least one detectable amplification molecule. A bipartite probe has a nucleic acid binding sequence capable of hybridizing the target genetic sequence in a sample of DNA and a binding probe sequence capable of hybridizing with a nucleic acid sequence of a detectable amplification molecule. An amplification molecule has a nucleic acid sequence capable of hybridizing the binding probe sequence of the bipartite probe. The amplification probe is detectable by a variety of methods.

More specifically, this invention provides a method for detection or quantitation of a target genetic sequence in a genetic sample with an assay which involves using: a bipartite probe having a target nucleic acid binding sequence capable of hybridizing the target genetic sequence and an nucleic acid binding probe sequence capable of hybridizing with a nucleic acid sequence of a primary detectable amplification molecule and a primary detectable amplification molecule having a nucleic acid sequence capable of hybridizing the binding probe sequence of the bipartite probe.

Additionally, this invention provides a method to increase signal strength in an assay for a target genetic sequence in a genetic sample involving the steps of: hybridizing at least one detectable amplification molecule with a bipartite probe in a hybridization solution, the bipartite probe having a target nucleic acid binding sequence capable of hybridizing the target genetic sequence and a binding probe sequence capable of hybridizing with a nucleic acid sequence of at least one detectable amplification molecule.

Similarly, this invention provides a method to increase signal strength in an assay for a target genetic sequence in a genetic sample involving the steps of: hybridizing primary and secondary amplification molecules in a hybridization solution and adding a bipartite probe. The bipartite probe having a target nucleic acid binding sequence capable of hybridizing the target genetic sequence and a binding probe sequence capable of hybridizing with a nucleic acid sequence of at least one detectable amplification molecule.

Additionally, this invention provides a method of forensic analysis for a target nucleic acid sequence involving the steps of: isolating human genomic DNA; immobilizing the human genomic DNA on a flat substrate; hybridizing the human genomic DNA with a bipartite probe having a target nucleic acid binding sequence capable of hybridizing the target genetic sequence and a binding probe sequence capable of hybridizing with a nucleic acid sequence of a primary detectable amplification molecule; and a primary detectable amplification molecule having a nucleic acid sequence capable of hybridizing the binding probe sequence of the bipartite probe; detecting the detectable amplification molecule; and correlating the detectable amplification molecule with the target nucleic acid sequence.

### BRIEF DESCRIPTION OF THE DRAWINGS

A more complete understanding of the invention and its advantages will be apparent from the following Description of the Preferred Embodiment(s) taken in conjunction with the accompanying drawings, wherein:

FIG. 1 is an illustration of the present method.

FIG. 2 is a graph showing percent formamide vs negative change in temperature.

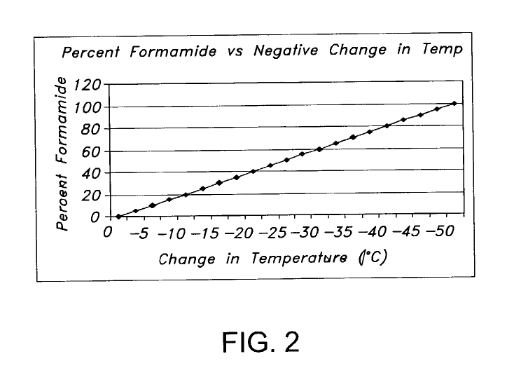

### DETAILED DESCRIPTION OF THE PREFERRED EMBODIMENT

The present invention provides a method for discriminating a genetic sequence using a bipartite probe to localize an amplification molecule. All patents, patent applications and articles discussed or referred to in this specification are hereby incorporated by reference.

The following terms and acronyms are used throughout the detailed description:

1. Definitions

complementary—chemical affinity between nitrogenous bases as a result of hydrogen bonding. Responsible for the base pairing between nucleic acid strands. Klug, W. S. and Cummings, M. R. (1997) Concepts of Genetics, 5^{th ed}., Prentice-Hall, Upper Saddle River, N.J. (hereby incorporated by reference)

dendrimer—synthetic 3-dimensional polymer macromolecule that is prepared from simple branched monomer units. The functionality of the macromolecule can be controlled and varied.

DNA (deoxyribonucleic acid)—The molecule that encodes genetic information. DNA is a double-stranded molecule held together by weak bonds between base pairs of nucleotides. The four nucleotides in DNA contain the bases: adenine (A), guanine (G) cytosine (C), and thymine (T). In nature, base pairs form only between A and T and between G and C; thus the base sequence of each single strand can be deduced from that of its partner.

genome—all the genetic material in the chromosomes of a particular organism; its size is generally given as its total number of base pairs.

genomic DNA—all of the genetic information encoded in a cell. Lehninger, A. L., Nelson, D. L. Cox, M. M. (1993) Principles of Biochemistry, 2^{nd} ed., Worth Publishers, New York, N.Y. (hereby incorporated by reference)

genotype—genetic constitution of an individual cell or organism.

microarray imager—is a reader used to detect samples bound or affixed to a flat substrate.

microarray technology—is a hybridization-based process that allows simultaneous quantitation of many nucleic acid species, has been described (M. Schena, D. Shalon, R. W. Davis, and P. O. Brown, “Quantititative Monitoring Of Gene Expression Patterns With A Complementary DNA Microarray,” Science, 270(5235), 467-70, 1995; J. DeRisi, L. Penland, P. O. Brown, M. L. Bittner, P. S. Meltzer, M. Ray, Y, Chen, Y. A. Su, and J. M. Trent, “Use Of A Cdna Microarray To Analyze Gene Expressions Patterns In Human Cancer,” Nature Genetics, 14(4), 457-60 (“DeRisi”), 1996; M. Schena, D. Shalon, R. Heller, A Chai, P. O. Brown, and R. W. Davis, “Parallel Human Genome Analysis: Microarray-Based Expression Monitoring Of 100 Genes,” Proc. Natl. Acad. Sci. USA., 93(20), 10614-9, 1996) hereby incorporated by reference. This technique combines robotic spotting of small amounts of individual, pure nucleic acids species on a glass surface, hybridization to this array with multiple fluorescently labeled nucleic acids, and detection and quantitation of the resulting fluor tagged hybrids with a scanning confocal microscope. This technology was developed for studying gene expression.

recombinant DNA—A combination of DNA molecules of different origin that are joined using recombinant DNA technologies.

substrate—Any three dimensional material to which sample or probe may be deposited that may have reactive groups to aid in attachment.

target genetic sequence—includes a transgenic insert, a selectable marker, recombinant site or any gene or gene segment.

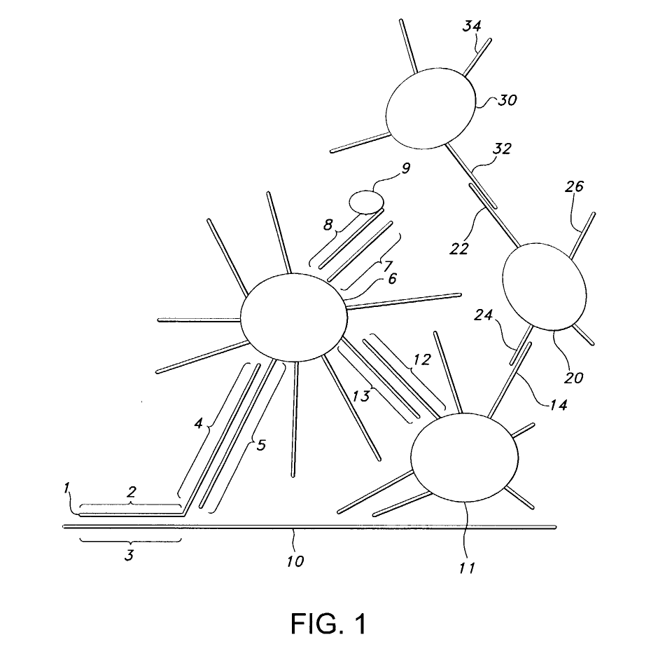

With reference to FIG. 1, the present invention is a process which indirectly detects or quantitates a target genetic sequence in any genetic sample 10. The genetic sample 10 can include genomic DNA, PCR amplicons, total RNA, mRNA, mitochondrial DNA, cDNA, plamid DNA, cosmid DNA and chloroplastic DNA. A bipartite probe 1 includes a target nucleic acid binding sequence 2 capable of hybridizing with the target genetic sequence 3 and an additional nucleic acid binding probe sequence 4 adjacent to the target nucleic acid binding sequence 2. A bipartite probe 1 is hybridized with both the genetic sample 10 including the target genetic sequence 3 and a detectable amplification molecule 6. The detectable amplification molecule 6 includes a capture probe sequence 5 that is a nucleic acid sequence capable of hybridizing with the nucleic acid binding probe sequence 4 of the bipartite probe 1. An amplification in molecule is any element which has the capacity to be detected while bound to a target. A nucleic acid sequence attached to an amplification molecule can be referred to as a capture arm. The capture probe sequence 5 is an example of a capture arm. A capture arm is preferably about 31 bases in length, but can range from about 16 to 90 bases in length. A capture arm to be capable of hybridization should be substantially linear in confirmation.

The primary detectable amplification molecule 6 can be a dendrimer that includes a label in its core or more commonly the label is on the dendrimer arms. The primary detectable amplification molecule 6 can also be a less branched or linear labeled or unlabeled oligonucleotides that can generate signal and/or facilitate the attachment of other labeled molecules. By way of example intermediary oligonucleotides may be hybridized to either the capture probe sequence or the binding probe sequence. Subsequently, this intermediary would bind to its remaining target. These intermediary oligonucleotides would not change the basic and novel aspects of the claimed method.

Moreover, a secondary signal generation molecule may also be added to the hybridization mixture to further increase detectable signal. Similarly, tertiary 20 and quaternary 30 etc. amplification molecules may successively be added to yield increase signal.

A plurality of nucleic acid binding sequences 7 and 13 can be attached to the primary amplification probe molecule 6. These nucleic acid binding sequence must have a specific affinity for the capture arm of another amplification probe molecule. The number of bases that compose the nucleic acid binding sequence can vary based on the size of the genome under study. The probe binding sequence 7 is capable of hybridizing with a single stranded genetic sequence 8 that includes a signal generating means 9. The single stranded genetic sequence 8 is hybridized to one of the capture arms of the primary detectable amplification molecule 6. This signal generating means 9 can include various labels. The label can be enzymes, radioactive isotopes, flurogenic, chemiluminescent or electrochemical materials, or members of a specific binding pair such as biotin-avidin, biotin-streptavidin, folic acid-folate binding proteins. Additionally, examples of covalent binding pairs include sulfhydryl reactive groups such as maleimides and haloacetyl derivatives and amine reactive groups such as isothio cynontes, succinimidyl esters, sulfonyl halides, and coupler esters, sulfonyl halides, and coupler dyes such as 3-methyl-2-benzothiazolinone hydrazone (MBTH) and 3-(dimethyl-amino) benzoic acid. The nucleic acid binding sequence 13 is one of the capture arms of the primary amplification probe molecule 6.

Additionally, a secondary detectable amplification molecule 8 and 11 can be used to further amplify the signal. The secondary amplification molecule 11 can be a secondary signal generating dendrimer with nucleic acid binding sequences 12 and 14. The nucleic acid binding sequences 12 and 14 are the capture arms of the secondary detectable amplification molecule 11. More specifically, the nucleic acid binding sequence 13 is capable of hybridizing with a nucleic acid binding sequence 12 attached to the secondary detectable amplification molecule 11. The nucleic acid binding sequence 12 can be referred to as the capture arm of the secondary detectable amplification molecule 11. It has been discovered that the linearity of the nucleic acid binding sequence 12 of the secondary genetic sequence attached to the secondary detectable amplification molecule 11 is a factor in detectable signal strength. Similarly, the linearity or lack of secondary structures is important for all nucleic acid sequences in the amplification reaction.

Additionally, to further increase signal strength a tertiary detectable amplification molecule 20, such as a tertiary signal generating dendrimer can be subsequently added to the hybridization solution. The tertiary amplification molecule 20 includes a nucleic acid binding sequence 24 that is capable of hybridizing with a nucleic acid binding sequence 14 attached to the secondary detectable amplification molecule 11. The nucleic acid binding sequences 24 and 26 of the tertiary amplification molecule 20 are the capture arms of the tertiary detectable amplification molecule 20. The tertiary detectable amplification molecule 20 also includes a nucleic acid sequence 22 that is capable of hybridizing with a nucleic acid sequence 32 attached to a quaternary detectable amplification molecule, such as a quaternary detectable amplification molecule 30. The quaternary detectable amplification molecule has a nucleic acid sequence 34 that is capable of binding an additional amplification molecule. The nucleic acid binding sequences 34 and 32 of the quaternary detectable amplification molecule 30 are the capture arms of the quaternary detectable amplification molecule 30. Additional signal generating molecules can be added until the amount of quantitated signal exceeds the maximum values the detection instrument can record.

For example, the nucleic acid binding probe sequence 4 of the bipartite probe 1 may have reverse compliment sequence as the 5′ end of reverse transcriptase. The bipartite probe 1 would contain the binding sequence of:

5′ CGC CGA CTC ACT GCG CGT CTT CTG TCC CGC C—Probe Sequence 3′. (SEQ ID NO: 1)

This nucleic acid binding probe sequence 4 of the bipartite probe 1 is complimentary to the nucleic acid sequence 5 for an amplification dendrimer probe from Genisphere (Hatfield, Pa.) 5′ CCG GCT GAG TGA CGC GCA GAA GAC AGG GAC G—3′. (SEQ ID NO: 2). A fluorescent dye is attached to the dendrimer core and/or the dendrimer arms. This fluorescent dye is a detectable label.

The target nucleic acid binding sequence 2 is specific for the target genetic sequence 3 of the genetic sample 10 respectively. The binding probe sequence 4 of the bipartite probe 1 is free and does not bind to the target genetic sequence 3. Examples of bipartite probes 1, complementary to single copy genes, are shown the table below:

TABLE 1

| | | |
|---|---|---|
| AAA32.MMHAP9FLC5. | 5′-GGC CGA CTC ACT GCG CGT CTT CTG TCC CGC | (SEQ ID NO: 3) |
| seq.53F | CATCACAAGTACTGGGAGAGG | |
| AAAMHAa67g1.seq.120 | 5′-GGC CGA CTC ACT GCG CGT CTT CTG TCC CGC | (SEQ ID NO: 4) |
| F | CGTCTCAGAGGTTAACTCACC | |
| | 5′-GGC CGA CTC ACT GCG CGT CTT CTG TCC CGC | (SEQ ID NO: 5) |
| | CTTCTTATCTTCAGCCCCACC | |
| AAAD9Mit211.1.38 | | |
| AAAX6134.129F | 5′-GGC CGA CTC ACT GCG CGT CTT CTG TCC CGC | (SEQ ID NO: 6) |
| | CATAACACGGTGTGCACCACG | |
| AAAU11075.95F | 5′-GGC CGA CTC ACT GCG CGT CTT CTG TCC CGC | (SEQ ID NO: 7) |
| | CTCCCTTCCTGTTGACTACAG | |
| AAAZ49987.38F | 5′-GGC CGA CTC ACT GCG CGT CTT CTG TCC CGC | (SEQ ID NO: 8) |
| | CTACCCACACGGGCTTAAAAC | |
| AAA32.MMHAP9FLC5. | 5′-GGC CGA CTC ACT GCG CGT CTT CTG TCC CGC | (SEQ ID NO: 9) |
| | CCACTGCCAGTGTGTTTTCAC | |

Examples of bipartite probes 1, complementary to Mouse Major Urinary Protein are shown in the table 2 below. These bipartite probes 1 are made one of the MUP genetic sequences, the target nucleic acid binding sequence 2 and a nucleic acid binding probe sequence 4, that is complementary to the nucleic acid sequence of an amplification molecule, which in this example is a dendrite probe.

TABLE 2

| | | |
|---|---|---|
| Mup_probe 1 | 5′-GGCCGACTCACTGCGCGTCTTCTGTCCCGCCCTGTGACGTATGATGGATTC | (SEQ ID NO: 10) |
| | AATACA | |
| Mup probe2 | 5′GGCCGACTCACTGCGCGTCTTCTGTCCCGCCTCGGCCATAGAGCTCCATC | (SEQ ID NO:11) |
| | AGCTGGA | |
| Mup probe 3 | 5′-GGCCGACTCACTGCGCGTCTTCTGTCCCGCCCTGTATGGATAGGAAGGGATGATG | (SEQ ID NO:12) |
| | C | |
| Mup probe4 | 5′-GGCCGACTCACTGCGCGTCTTCTGTCCCGCCGGCTCAGGCCATTCTTCATTCTCGG | (SEQ ID NO:13) |
| GCCT. | | |

An amplification molecule, such as a dendrimer or tyramide, is introduced. The amplification molecule is bound directly or indirectly to the bipartite. The amplification molecules, such as a dendrite probes, has a nucleic acid capture probe sequence 5 that is complementary to the nucleic acid binding probe sequence 4 of the bipartite probe. The bipartite probe 1 complexed with nucleic acid capture probe sequence 5 of the amplification molecule may be hybridized to a secondary signal generating molecule, such as an additional dendrimers with complimentary sequences or a secondary labeled oligonucleotide such as nucleic acid sequence 8.

Free bipartite probes and excess amplification molecules are removed via several successive wash steps. The bound detectable amplification molecule emits a detectable signal that can have a linear relationship to the number of bound molecules.

One modification of the binding probe sequence 4 of the bipartite probe 1 and/or the capture probe sequences of any of dendrites capture probe sequences include, but not limited to biotinylation. A detectable amplification molecule may have a signal molecule directly attached to it or it may activate or facilitate the attachment of another signal unit. An example of a secondary signal generation element includes but not limited to enzymes and labeled antibodies. Multiple signals units may be used to amplify the signal of the target.

The bipartite probe 1, which is composed of the target nucleic acid binding sequence 2 and the binding probe sequence 4, is added to a genetic sample 10, preferably genomic DNA. The target nucleic acid binding sequence 2 of the bipartite probe 1 specifically hybridizes to the target genetic sequence 3 of the genomic DNA sample 10. The nucleic acid binding sequence 5 of the amplification probe molecule 6 specifically hybridizes to the binding probe sequence 4 elements of the bipartite probe 1. In one embodiment the primary detectable amplification molecule 6 is a dendrimer and includes a fluorescent tag that functions as a label in an assay. The signal may further be increased by subsequently adding various forms of secondary, tertiary, quaternary etc signal generating units. In an alternative embodiment the amplification molecules are hybridized together prior to adding the bipartite probe 1.

Alternatively, the modification, such as proteins (eg. Biotinylation), may be added to either the binding probe sequence of the bipartite and/or the capture probe sequence of the dendrite. This modification will allow for protein interaction resulting in indirect labeling.

In the preferred embodiment DNA from a genetic sample is immobilized onto a substrate, preferably a glass slide. The substrates typically have a reactive group attached to the surface to facilitate the immobilization. Aldehyde reactive groups covalently coupled to the glass subsequently covalently immobilize the DNA via a dehydration reaction. Typically, to reduce the background signal a Sodium Borohydrate Hydrate solution is added to reduce the unused aldehyde reactive groups to alcohol. The bound DNA is denatured via heating the substrate above 94° C. for five minutes. This heating process disrupts the hydrogen bonds between adenine, cytosine, guanine and thymine. A bipartite probe 1 is added to the substrate suspended in a hybridization buffer and covered with a coverslip. The bipartite probe 1 in solution is incubated at 43-55° C. for one hour. During this incubation the target nucleic acid genetic sequence 2 of the bipartite probe 1 specifically hybridizes to the target genetic sequence of the sample DNA 10. A three minute wash in 2× Saline Sodium Citrate (SSC) and a one minute wash in 0.2×SSC removes any unbound bipartite. Amplification molecules are suspended in a hybridization buffer and added to the substrate and coverslipped. The amplification molecules in solution are incubated at 50-55° C. for one hour. During this incubation the binding probe sequence of the bipartite probe 1 specifically hybridizes to the capture arm of the amplification probe molecule. A three minute wash in 2×SSC and a one minute wash in 0.2×SSC removes any unbound amplification probe molecules. The secondary signal generating dendrite probe, has a nucleic acid capture probe sequence that is complementary to the capture probe sequence of the primary detectable amplification probe 6. The secondary signal generating dendrite probes are suspended in a hybridization buffer. The “secondary signal generating dendrite” probe in solution are incubated at 50-55° C. for one hour. During this incubation the capture arm sequence of the secondary signal generating dendrite probe specifically hybridizes to the capture arm of the amplification probe molecule. A 15 minute wash in 2×SSC, 0.2% SDS, followed by three five minute washes in 2×SSC and a one minute wash in a 0.2×SSC removes any unbound molecules. In the preferred embodiment amplification molecules are added sequentially, however prehybridizing two or more amplification molecules together before hybridizing to the binding probe sequence of the bipartite is also contemplated.

A secondary signal generating dendrite probe serving as a secondary amplification molecule are suspended in a hybridization buffer. The secondary signal generating dendrite probe, has a nucleic acid binding sequence that is complementary to the nucleic acid binding sequence of the primary detectable amplification molecule 6. The bipartite probe 1 is complexed with capture probe sequences of the primary amplification molecule which may be complexed to a secondary amplification molecule.

Certain factors are considered when designing the capture arms as used in this invention. The first consideration is to make sure the sequence is specific for the genetic target in the genome under study. The four bases (adenine, thymine, guanine and cytosine) raised to the number of bases in the sequence (eg. 4^{number of bases}) should be a number larger than the number of bases in the genome being studied. By way of example, the number of bases in the mouse genome is 3.0×10⁹. For consideration of a bipartite's target nucleic acid binding sequence being 16 bases in length, the calculation would be 4¹⁶ which is 4.29×10⁹. Whereas a target nucleic acid binding sequence of 15 bases would yield 4¹⁵ which is 1.07×10⁹. This is less than 3.0×10⁹ rendering 15 bases inadequate to be discriminatory for the mouse genome.

The optimal hybridization temperature is dependent on the length and composition of the oligonucleotide. The melting temperature of each element determines the optimal hybridization of both the target nucleic acid binding sequence and the nucleic acid binding probe sequence of the bipartite probe. Each melting temperature is determined independently.

By way of example the melting temperature of a 20 mer target nucleic acid binding sequence is 68.3° C. A 20 mer is long enough to confer specificity to the eukaryotic genome of 3.0 10⁹ base pairs. The optimal hybridization temperature is 20-25° C. lower than 68.3° C. resulting in a 43.0° C. hybridization temperature with a tradition buffer such as (1) 0.25 NaPO₄, 4.5% SDS ImM EDTA, 1×SSC, 2× Denhardt's solution or (2) 2×SSC, 1% SDS. The same 20 mer target nucleic acid binding sequence with the 68.3° C. melting temperature in a 40 % formamide buffer has a remarkably different optimal hybridization temperature. In addition to the lower temperature of 20-25° C. and additional decrease of 20° C. is required resulting in an optimal hybridization temperature of 23.0° C.

The nucleic acid binding probe sequence 4 of the bipartite probe 1 is also determined in the same fashion. The nucleic acid binding probe sequence 4 for a 31 mer has a melting temperature of 94.0° C. For traditional hybridization buffers 20-25° C. below the melting temperate results in an optimal temperature of 70.0° C. In a 40% formamide solution the optimal hybridization temperature decreases 20° C. resulting in a hybridization temperature of 50.0° C.

A 16 mer target nucleic acid binding probe sequence 4 is the minimum number of bases to confer specificity to the eukarotic genome. A 16 mer has a melting temperature of 54.0° C. Traditional, hybridization buffers optimally hybridizing at 20° C. less than the melting temperature yields optimal results at 29.0° C. Whereas a 40% formamide buffer requires an additional 20° C. decrease in temperature to optimal hybridize at 9.0° C.

The nucleic acid binding probe sequence 4 for a 31 mer has a melting temperature of 94.0° C. 20-25° C. below the melting temperate is the traditional range for optimal hybridization. For the 31 mer a traditional hybridization buffer has an optimal hybridization temperature of 70.0° C. In a 40% formamide solution the optimal hybridization temperature decreases 20° C. resulting in a hybridization temperature of 50.0° C.

The number of bases to confer specificity to a prokaryote with a genome of eighteen thousand base pairs is less than the number of bases to be discriminatory for the eukaryotic genome. As an example, a 8 mer has enough bases to be discriminatory for a genome of eighteen thousand base pairs. An 8 mer target nucleic acid binding sequence 2 will have a low melting temperature due to few number of bases. An 8 mer with a melting temperature of 27.0° C. in a traditional buffer has a realized optimal hybridization at 2.0° C. Because of the 20° C. drop in temperature below that of a traditional buffer, a formamide hybridization of an 8 mer is not possible in formamide.

A 13 mer target nucleic acid binding sequence 2 is long enough to confer specificity to a prokaryotic genome of eighteen thousand base pairs. A 13 mer with a melting temperature of 45.0° C. will optimally hybridize in a traditional buffer at 19.0° C. Whereas it is possible to optimally hybridize in 40% formamide at 0° C.

Therefore the preferred number of bases for the target nucleic acid binding sequence 2 is 16-20 bases for the eukarotic genome. Whereas by contrast the preferred length of the target nucleic acid binding sequence 2 is 8-13 bases for prokaryotes with a genome of 18,000 base pairs.

The preferred number of bases for the nucleic acid binding probe sequence 4 is for eukaryotes and prokaryotes is the same. A preferred length of 31 bases confers specifically to its compliment.

A sequence search, known as a blast from public internet sites such as National Center for Biotechnology Information (NCIB) available at http://www.ncbi.nlm.nih.gov/ or private databases such as Celera's (Rockville, Md.) should be performed. A blast of all known sequences for a particular species helps ensure specificity. If a blast reveals that a particular sequence exists in the genome then no preference will be appreciable between the target sequence and the endogenous sequence.

Because of the hydrogen bonding between adenine/thymine and guanine/cytosine, two and three bonds respectively, secondary structures can form. The secondary structures include stem loop structures and homodimers. Stem loop structures form because of the affinity of one region of the bipartite for another region of the same bipartite, specifically between the guanine and cytosine residues. Whereas homodimer show an affinity for anther molecule of the same species.

In order to provide a capture arm capable of hybridization both of these secondary structures should be eliminated in a variety of ways. Simply selecting an alternative sequence that is not rich in guanine and cytosine often is all that is needed to produce a species with no stable secondary structures. If the sequence is sufficiently long enough, it is possible to delete the portion of the arm that participates in the formation of the secondary structure as long as the remaining element is sufficient for specific hybridization. The application of heat sufficient to overcome the secondary structures is highly effective. However, the temperature to optimally hybridize should be higher than the temperature to reduce secondary structures.

Secondary structures may also be reduced by the substitution of bases. Inosine is a nucleoside that can be substituted for a base participating in the formation of a secondary structure thereby destabilizing the structure. Moreover, substituting a base that forms a mismatch will also destabilize the secondary structures. Although, there must be sufficient base pairing between the capture arms and the target to yield adequate hybridization in spite of the mismatch.

By way of example, the 29^{th} base, which is guanine, was substituted with adenine in order to disrupt the stem loop structure in a secondary signal generating dendrimer. This base mismatch destabilized the stein loop structure, which then allows nucleic acid arm to participate in a hybridization reaction. The melting temperature to disassociate the hairpin structure because of the base mismatch was 7° C. as opposed to 70° C. for the perfect match. However, the mismatch does not prevent hybridization between amplification molecule and the secondary signal generating dendrimer.

The secondary signal generating dendrimer had a capture sequence of:

5′—GGC CGA CTC ACT GCG CGT CTT CTG TCC CAC C—3′ (SEQ ID NO: 14) which had the following characteristics:

TABLE 3
COMPOSITION:

| | | |
|---|---|---|
| A | 3.00 | 9.7% |
| C | 14.00 | 45.2% |
| G | 7.00 | 22.6% |
| T | 7.00 | 22.6% |
| X | .00 | 0.0% |
| A + T | 10.00 | 32.3% |
| C + G | 21.00 | 67.7% |

STEM LOOP STRUCTURE:

| | |
|---|---|
| CTCAGCCGG 5′ | (SEQ ID NO:15) |
| < ∥ | |
| ACTGCGCGTCTTCTGTCCCACC 3′ | (SEQ ID NO:16) |

G=0.5 kcal/mol

loop Tm=7° C.

HOMODIMER:

(SEQ ID NO:17)

5′GGCCGACTCACTGCGCGTCTTCTGTCCCACC 3′

(SEQ ID NO:18)

3′CCACCCTGTCTTCTGCGCGTCACTCAGCCGG 5′

Homodimer Tm=32.7° C.

The sequence 5′ GGC CGA CTC ACT GCG CGT CTT CTG TCC CGC C 3′ (SEQ ID NO: 19) is the perfect match for the capture probe sequence of the amplification molecule however it has the following characteristics:

TABLE 4
COMPOSITION:

| | | |
|---|---|---|
| A | 2.00 | 6.5% |
| C | 14.00 | 45.2% |
| G | 8.00 | 25.8% |
| T | 7.00 | 22.6% |
| X | .00 | 0.0% |
| A + T | 9.00 | 29.0% |
| C + G | 22.00 | 71.0% |

| | |
|---|---|
| STEM LOOP STRUCTURE: | |
| \| | |
| CTTCTGCGCGTCACTCAGCCGG 5′ | (SEQ ID NO:20) |
| < ∥\| | |
| TGTCCCGCC 3′ | (SEQ ID NO:21) |
| \| | |

G=−2.4 kcal/mol

loop Tm=70° C.

HOMODIMER:

(SEQ ID NO:22)

5′GGCCGACTCACTGCGCGTCTTCTGTCCCGCC 3′

(SEQ ID NO:23)

3′CCGCCCTGTCTTCTGCGCGTCACTCAGCCGG 5′

Homodimer Tm=34.0° C.

Another alternative for destabilizing the loop structure and affecting the optimal hybridization temperature is the addition of a chemical agent such as formamide. Formamide changes the melting temperature and expected optimal hybridization temperate one degree Celsius for every 2% formamide added beyond the traditionally accepted optimal hybridization range of 20-25° C. below the melting temperature. Therefore adjusting the percentage can dramatically affect the thermal requirements for a successful reaction.

The Binding Probe Sequence of the bipartite which is:

5′—GGC CGA CTC ACT GCG CGT CTT CTG TCC CGC C—3′ (SEQ ID NO:24) with the following characteristics:

TABLE 5
COMPOSITION:

| | | |
|---|---|---|
| A | 2.00 | 6.5% |
| C | 14.00 | 45.2% |
| G | 8.00 | 25.8% |
| T | 7.00 | 22.6% |
| X | .00 | 0.0% |
| A + T | 9.00 | 29.0% |
| C + G | 22.00 | 71.0% |

STEM LOOP STRUCTURE:

| | |
|---|---|
| \| | |
| CTTCTGCGCGTCACTCAGCCGG 5′ | (SEQ ID NO:25) |
| < ∥\| | |
| TGTCCCGCC 3′ | (SEQ ID NO:26) |
| \| | |

G=−2.4 keal/mol

loop Tm=70° C.

HOMODIMER:

(SEQ ID NO:27)

5′GGCCGACTCACTGCGCGTCTTCTGTCCCGCC 3′

(SEQ ID NO:28)

3′CCGCCCTGTCTTCTGCGCGTCACTCAGCCGG 5′

Homodimer Tm=34.0° C.

### EXAMPLE 1

Bipartite Probe and Single Amplification Probe Molecule

Synthetic single stranded DNA oligonucleotides 51 mers bipartites were synthesized by Invitrogen (Carlsbad, Calif.). The dry bipartites were rehydrated with deionized water at a concentration of 200 μM. The bipartites mixture consisting of 1 μl of bipartite, 4.5 μl of 20×SSC (Saline Sodium Citrate) and 24.5 μl of deionized water was added to the wells of a Whatman (Clifton, N.J.) polypropylene 384, 80 μl V-bottom wellplate. The bipartites were printed with a SpotBot (Telechem, Sunnyvale, Calif.) onto Superaldehyde substrates (Telechem, Sunnyvale, Calif.) in a grid pattern of five columns by six rows. The printed substrate is loaded into a heating cassette. The heating cassette is composed of a beveled top plate, prefabricated spacers, a metal frame and tension clips. The substrate is lowered into the metal frame and spacers are placed on top of the substrate running lengthwise along the edge. The beveled top plate is then lowered on top of the substrate only separated by the spacers. The metal tension clips are then applied to the heating cassette, which holds the cassette together securely.

The heating cassette is placed on the exterior platform of the heating block. The heating block's exterior surface is thermally controlled by different temperature fluids being perfused by external circulator baths. The contact between the heating cassette substrate and the heating block permits a highly efficient thermal transfer.

The slide was then washed six times with 350 μl of deionized water. The remaining reactive aldehyde groups were removed from the slide by washing the slide six times with 350 μl of Sodium Borohydrate (0.4 g NaBH₄, 10 mls 100% ethanol, 30 mls of PBS) followed by six washes with 350 μl of deionized water. 4 μl of CY3 dendrimer (primary detectable amplification Molecule) and 116 μl of 40 % Formamide, 4×SSC, 1% SDS, 2′ Denhardt's Solution, was incubated at 50° C. for 60 minutes. The substrate was washed seven times with 350 μl of 2′ SSC, 0.2% SDS, followed by eleven 350 μl washes in 2×SSC and two 350 μl washes in a 0.2×SSC. The slides was dried and scanned at a gain of 255, 200 and 100. The following results from 60 samples demonstrate a single amplification molecule attached to a bipartite and can be used as a baseline for additional amplifications.

TABLE 6

| 255 | | | 200 | | | 100 | | |
|---|---|---|---|---|---|---|---|---|
| | | Baseline | | | Baseline | | | Baseline |
| | Position | Density | | Position | Density | | Position | Density |
| 1 | 1-1:1 | 6941.05 | 1 | 1-1:1 | 875.48 | 1 | 1-1:1 | 0.33 |
| 2 | 1-1:2 | 8346.22 | 2 | 1-1:2 | 1053.07 | 2 | 1-1:2 | 0.28 |
| 3 | 1-1:3 | 5868.25 | 3 | 1-1:3 | 658.26 | 3 | 1-1:3 | 1.01 |
| 4 | 1-1:4 | 4551.32 | 4 | 1-1:4 | 497.52 | 4 | 1-1:4 | 9 |
| 5 | 1-1:5 | 5146.45 | 5 | 1-1:5 | 575.44 | 5 | 1-1:5 | 4.74 |
| 6 | 1-2:1 | 1798.24 | 6 | 1-2:1 | 197.07 | 6 | 1-2:1 | 3.99 |
| 7 | 1-2:2 | 3528.95 | 7 | 1-2:2 | 641.32 | 7 | 1-2:2 | 4.94 |
| 8 | 1-2:3 | 942.91 | 8 | 1-2:3 | 115 | 8 | 1-2:3 | 5.11 |
| 9 | 1-2:4 | 886.56 | 9 | 1-2:4 | 96.89 | 9 | 1-2:4 | 12 |
| 10 | 1-2:5 | 1076.32 | 10 | 1-2:5 | 122.1 | 10 | 1-2:5 | 8.92 |
| 11 | 1-3:1 | 1372.14 | 11 | 1-3:1 | 149.66 | 11 | 1-3:1 | 9.16 |
| 12 | 1-3:2 | 900.74 | 12 | 1-3:2 | 103.67 | 12 | 1-3:2 | 9.55 |
| 13 | 1-3:3 | 3606.58 | 13 | 1-3:3 | 412.24 | 13 | 1-3:3 | 4.39 |
| 14 | 1-3:4 | 15767.1 | 14 | 1-3:4 | 2431.13 | 14 | 1-3:4 | 7.77 |
| 15 | 1-3:5 | 11082.13 | 15 | 1-3:5 | 1418.62 | 15 | 1-3:5 | 0.88 |
| 16 | 1-4:1 | 9801.71 | 16 | 1-4:1 | 1232.66 | 16 | 1-4:1 | 9.19 |
| 17 | 1-4:2 | 12067.38 | 17 | 1-4:2 | 1466.64 | 17 | 1-4:2 | 7.31 |
| 18 | 1-4:3 | 11162.8 | 18 | 1-4:3 | 1500.59 | 18 | 1-4:3 | 6.05 |
| 19 | 1-4:4 | 18770.91 | 19 | 1-4:4 | 3066.03 | 19 | 1-4:4 | 2.57 |
| 20 | 1-4:5 | 16521.03 | 20 | 1-4:5 | 2376.26 | 20 | 1-4:5 | 7.48 |
| 21 | 1-5:1 | 18222.83 | 21 | 1-5:1 | 2551.4 | 21 | 1-5:1 | 0.31 |
| 22 | 1-5:2 | 17484.73 | 22 | 1-5:2 | 2576.47 | 22 | 1-5:2 | 0.66 |
| 23 | 1-5:3 | 11803.16 | 23 | 1-5:3 | 1483.21 | 23 | 1-5:3 | 0.2 |
| 24 | 1-5:4 | 14515.22 | 24 | 1-5:4 | 2174.71 | 24 | 1-5:4 | 0.22 |
| 25 | 1-5:5 | 3346.52 | 25 | 1-5:5 | 379.09 | 25 | 1-5:5 | 0.23 |
| 26 | 1-6:1 | 17863.92 | 26 | 1-6:1 | 2570.57 | 26 | 1-6:1 | 0.25 |
| 27 | 1-6:2 | 14544.42 | 27 | 1-6:2 | 2053.7 | 27 | 1-6:2 | 0.2 |
| 28 | 1-6:3 | 12623.67 | 28 | 1-6:3 | 1787.79 | 28 | 1-6:3 | 0.26 |
| 29 | 1-6:4 | 7320.88 | 29 | 1-6:4 | 889.35 | 29 | 1-6:4 | 0.17 |
| 30 | 1-6:5 | 14564.03 | 30 | 1-6:5 | 2016.93 | 30 | 1-6:5 | 0.23 |
| 31 | 2-1:1 | 987.97 | 31 | 2-1:1 | 106.75 | 31 | 2-1:1 | 1.27 |
| 32 | 2-1:2 | 1354.89 | 32 | 2-1:2 | 149.03 | 32 | 2-1:2 | 0.4 |
| 33 | 2-1:3 | 1871.3 | 33 | 2-1:3 | 200.87 | 33 | 2-1:3 | 2.41 |
| 34 | 2-1:4 | 3288.86 | 34 | 2-1:4 | 363.07 | 34 | 2-1:4 | 5.95 |
| 35 | 2-1:5 | 5657.32 | 35 | 2-1:5 | 782.71 | 35 | 2-1:5 | 6.52 |
| 36 | 2-2:1 | 4965.23 | 36 | 2-2:1 | 568.64 | 36 | 2-2:1 | 6.8 |
| 37 | 2-2:2 | 5781.66 | 37 | 2-2:2 | 726.88 | 37 | 2-2:2 | 5.98 |
| 38 | 2-2:3 | 4639.2 | 38 | 2-2:3 | 528.22 | 38 | 2-2:3 | 5.43 |
| 39 | 2-2:4 | 7146.23 | 39 | 2-2:4 | 906.97 | 39 | 2-2:4 | 5.24 |
| 40 | 2-2:5 | 797.15 | 40 | 2-2:5 | 90.35 | 40 | 2-2:5 | 5.25 |
| 41 | 2-3:1 | 3339.81 | 41 | 2-3:1 | 417.37 | 41 | 2-3:1 | 3.43 |
| 42 | 2-3:2 | 1965.02 | 42 | 2-3:2 | 204.02 | 42 | 2-3:2 | 1.34 |
| 43 | 2-3:3 | 7152.04 | 43 | 2-3:3 | 864.81 | 43 | 2-3:3 | 2.13 |
| 44 | 2-3:4 | 10749.06 | 44 | 2-3:4 | 1751.83 | 44 | 2-3:4 | 2.06 |
| 45 | 2-3:5 | 10882.52 | 45 | 2-3:5 | 1821.55 | 45 | 2-3:5 | 1.77 |
| 46 | 2-4:1 | 12678.6 | 46 | 2-4:1 | 1919.5 | 46 | 2-4:1 | 7.66 |
| 47 | 2-4:2 | 11469.72 | 47 | 2-4:2 | 1743.12 | 47 | 2-4:2 | 7.06 |
| 48 | 2-4:3 | 11576.24 | 48 | 2-4:3 | 1632.25 | 48 | 2-4:3 | 6.41 |
| 49 | 2-4:4 | 10454.25 | 49 | 2-4:4 | 1546.36 | 49 | 2-4:4 | 4.95 |
| 50 | 2-4:5 | 11237.03 | 50 | 2-4:5 | 1608.98 | 50 | 2-4:5 | 3.2 |
| 51 | 2-5:1 | 9560.81 | 51 | 2-5:1 | 1087.62 | 51 | 2-5:1 | 0.24 |
| 52 | 2-5:2 | 6008.46 | 52 | 2-5:2 | 633.04 | 52 | 2-5:2 | 0.23 |
| 53 | 2-5:3 | 8380.78 | 53 | 2-5:3 | 899.31 | 53 | 2-5:3 | 0.22 |
| 54 | 2-5:4 | 8412.45 | 54 | 2-5:4 | 895.45 | 54 | 2-5:4 | 0.26 |
| 55 | 2-5:5 | 8094.66 | 55 | 2-5:5 | 840.9 | 55 | 2-5:5 | 0.22 |
| 56 | 2-6:1 | 16528.55 | 56 | 2-6:1 | 2198.41 | 56 | 2-6:1 | 0.26 |
| 57 | 2-6:2 | 15772.75 | 57 | 2-6:2 | 2045.09 | 57 | 2-6:2 | 0.22 |
| 58 | 2-6:3 | 14347.99 | 58 | 2-6:3 | 1881.88 | 58 | 2-6:3 | 0.21 |
| 59 | 2-6:4 | 10955.43 | 59 | 2-6:4 | 1439.66 | 59 | 2-6:4 | 0.2 |
| 60 | 2-6:5 | 9306.89 | 60 | 2-6:5 | 1075.13 | 60 | 2-6:5 | 0.18 |
| Mean | | 8463.18 | Mean | | 1140.04 | Mean | | 3.42 |
| SD | | 5312.07 | SD | | 800.15 | SD | | 3.32 |
| Min | | 797.15 | Min | | 90.35 | Min | | 0.17 |
| Max | | 18770.91 | Max | | 3066.03 | Max | | 12 |
| Range | | 17973.76 | Range | | 2975.68 | Range | | 11.83 |
| Sum | | 507791.01 | Sum | | 68402.64 | Sum | | 204.93 |

### EXAMPLE 2

Bipartite Probe and Multiple Amplification Probe Molecule

Synthetic single stranded DNA oligonucleotides 51 mers bipartites were synthesized by Invitrogen (Carlsbad, Calif.). The dry bipartites were rehydrated with deionized water at a concentration of 200 μM. The bipartites mixture consisting of 1 μl of bipartite, 4.5 μl of 20×SSC (Saline Sodium Citrate) and 24.5 μl of deionized water was added to the wells of a Whatman (Clifton, N.J.) polypropylene 384, 80 μl V-bottom wellplate. The bipartites were print with a SpotBot (Telechem, Sunnyvale, Calif.) onto Superaldehyde substrates (Telechem, Sunnyvale, Calif.) in a grid pattern of five columns by six rows. The printed substrate is loaded into a heating cassette. The heating cassette is composed of a beveled top plate, prefabricated spacers, a metal frame and tension clips. The substrate is lowered into the metal frame and spacers are placed on top of the substrate running lengthwise along the edge. The beveled top plate is then lowered on top of the substrate only separated by the spacers. The metal tension clips are then applied to the heating cassette, which holds the cassette together securely.

The heating cassette is placed on the exterior platform of the heating block. The heating block's exterior surface is thermally controlled by different temperature fluids being perfused by external circulator baths. The contact between the heating cassette substrate and the heating block permits a highly efficient thermal transfer. The slide was then washed six times with 350 μl of deionized water. The remaining reactive aldehyde groups were removed from the slide by washing the slide six times with 350 μl of Sodium Borohydrate (0.4 g NaBH_{4, 10}mls 100% ethanol, 30mls of PBS) followed by six washes with 350 μl of deionized water. 1 μl of CY3 dendrimer (Amplification Probe Molecule) and 116 μl of 40% Formamide, 4×SSC, 1% SDS, 2× Denhardt's Solution, was incubated at 55° C. for 60 minutes. The substrate was washed three times with 350 μl of 2×SSC followed by two 350 μl washes in 0.2×SSC. 1.5 μl of CY3 secondary signal generating dendrimer with the capture sequence of 5′—GGC CGA CTC ACT GCG CGT CTT CTG TCC CAC C—3′ (SEQ ID NO: 29) and 114 μl of 40% Formamide, 4×SSC, 1% SDS, 2× Denhardt's Solution, was incubated at 55° C. for 60 minutes. The substrate was washed seven times with 350 μl of 2×SSC, 0.2% SDS, followed by eleven 350 μl washes in 2×SSC and two 350 μl washes in a 0.2×SSC to remove any excess dendrimer. The slides was dried and scanned at a gain of 255, 200 and 100. The following results from 60 samples demonstrate an amplification of signal from a secondary signal generating dendrimers hybridized to an amplification probe molecule which in turn is hybridized to a bipartite. The secondary signal enhancement represented indicates a three to seven fold increase in signal over the baseline single bipartite and amplification probe molecule hybridization.

TABLE 7

| 255 | | | 200 | | | 100 | | |
|---|---|---|---|---|---|---|---|---|
| | | Enhancer | | | Enhancer | | | Enhancer |
| | Position | Density | | Position | Density | | Position | Density |
| 1 | 1-1:1 | 21466.58 | 1 | 1-1:1 | 3284.9 | 1 | 1-1:1 | 17.33 |
| 2 | 1-1:2 | 20608.05 | 2 | 1-1:2 | 2695.86 | 2 | 1-1:2 | 13.7 |
| 3 | 1-1:3 | 21356.09 | 3 | 1-1:3 | 2746.01 | 3 | 1-1:3 | 14.09 |
| 4 | 1-1:4 | 20785.57 | 4 | 1-1:4 | 3285.38 | 4 | 1-1:4 | 17.17 |
| 5 | 1-1:5 | 22079.76 | 5 | 1-1:5 | 2812.97 | 5 | 1-1:5 | 14.46 |
| 6 | 1-2:1 | 22249.47 | 6 | 1-2:1 | 3699.86 | 6 | 1-2:1 | 19.92 |
| 7 | 1-2:2 | 22943.9 | 7 | 1-2:2 | 3790.91 | 7 | 1-2:2 | 20.61 |
| 8 | 1-2:3 | 24759.33 | 8 | 1-2:3 | 3901.18 | 8 | 1-2:3 | 20.68 |
| 9 | 1-2:4 | 23492.35 | 9 | 1-2:4 | 3578.82 | 9 | 1-2:4 | 19.21 |
| 10 | 1-2:5 | 24550.58 | 10 | 1-2:5 | 3739.15 | 10 | 1-2:5 | 19.99 |
| 11 | 1-3:1 | 25725.09 | 11 | 1-3:1 | 5183.08 | 11 | 1-3:1 | 28.68 |
| 12 | 1-3:2 | 25157.78 | 12 | 1-3:2 | 4993.68 | 12 | 1-3:2 | 27.26 |
| 13 | 1-3:3 | 24589.76 | 13 | 1-3:3 | 4725.14 | 13 | 1-3:3 | 26.11 |
| 14 | 1-3:4 | 25405.29 | 14 | 1-3:4 | 5138.36 | 14 | 1-3:4 | 28.16 |
| 15 | 1-3:5 | 26018.43 | 15 | 1-3:5 | 4893.9 | 15 | 1-3:5 | 26.56 |
| 16 | 1-4:1 | 25912.36 | 16 | 1-4:1 | 5163.63 | 16 | 1-4:1 | 28.26 |
| 17 | 1-4:2 | 25761.34 | 17 | 1-4:2 | 5585.81 | 17 | 1-4:2 | 30.63 |
| 18 | 1-4:3 | 26454.03 | 18 | 1-4:3 | 5312 | 18 | 1-4:3 | 29.22 |
| 19 | 1-4:4 | 26585.17 | 19 | 1-4:4 | 5299.24 | 19 | 1-4:4 | 29.22 |
| 20 | 1-4:5 | 26444.09 | 20 | 1-4:5 | 5237.46 | 20 | 1-4:5 | 28.79 |
| 21 | 1-5:1 | 25935.77 | 21 | 1-5:1 | 5609.51 | 21 | 1-5:1 | 30.98 |
| 22 | 1-5:2 | 26376.21 | 22 | 1-5:2 | 5850.5 | 22 | 1-5:2 | 32.25 |
| 23 | 1-5:3 | 26816.98 | 23 | 1-5:3 | 5333.97 | 23 | 1-5:3 | 29.74 |
| 24 | 1-5:4 | 27171.67 | 24 | 1-5:4 | 5443.19 | 24 | 1-5:4 | 30.31 |
| 25 | 1-5:5 | 25551.35 | 25 | 1-5:5 | 4786.85 | 25 | 1-5:5 | 26.45 |
| 26 | 1-6:1 | 26995.04 | 26 | 1-6:1 | 7323.1 | 26 | 1-6:1 | 41.06 |
| 27 | 1-6:2 | 27327.11 | 27 | 1-6:2 | 5566.61 | 27 | 1-6:2 | 30.98 |
| 28 | 1-6:3 | 27168.12 | 28 | 1-6:3 | 5778.9 | 28 | 1-6:3 | 31.72 |
| 29 | 1-6:4 | 27293.74 | 29 | 1-6:4 | 5766.32 | 29 | 1-6:4 | 32.06 |
| 30 | 1-6:5 | 26982.91 | 30 | 1-6:5 | 5813.67 | 30 | 1-6:5 | 32.11 |
| 31 | 2-1:1 | 17940.81 | 31 | 2-1:1 | 2518.41 | 31 | 2-1:1 | 12.85 |
| 32 | 2-1:2 | 18939.96 | 32 | 2-1:2 | 2346.19 | 32 | 2-1:2 | 11.51 |
| 33 | 2-1:3 | 20290.34 | 33 | 2-1:3 | 2839.18 | 33 | 2-1:3 | 14.73 |
| 34 | 2-1:4 | 20495.33 | 34 | 2-1:4 | 2565.22 | 34 | 2-1:4 | 13 |
| 35 | 2-1:5 | 21312.59 | 35 | 2-1:5 | 3200.48 | 35 | 2-1:5 | 16.73 |
| 36 | 2-2:1 | 20425.43 | 36 | 2-2:1 | 2900.59 | 36 | 2-2:1 | 15.03 |
| 37 | 2-2:2 | 21927.46 | 37 | 2-2:2 | 2797.39 | 37 | 2-2:2 | 14.28 |
| 38 | 2-2:3 | 19091 | 38 | 2-2:3 | 2501.09 | 38 | 2-2:3 | 12.87 |
| 39 | 2-2:4 | 21339.34 | 39 | 2-2:4 | 2635.29 | 39 | 2-2:4 | 13.43 |
| 40 | 2-2:5 | 21733.1 | 40 | 2-2:5 | 2670.43 | 40 | 2-2:5 | 13.84 |
| 41 | 2-3:1 | 24060.99 | 41 | 2-3:1 | 3765 | 41 | 2-3:1 | 20.08 |
| 42 | 2-3:2 | 22036.33 | 42 | 2-3:2 | 3211.25 | 42 | 2-3:2 | 17.04 |
| 43 | 2-3:3 | 22234.28 | 43 | 2-3:3 | 3303.46 | 43 | 2-3:3 | 17.39 |
| 44 | 2-3:4 | 23778.08 | 44 | 2-3:4 | 3371.82 | 44 | 2-3:4 | 17.9 |
| 45 | 2-3:5 | 24821.74 | 45 | 2-3:5 | 3442.73 | 45 | 2-3:5 | 18.32 |
| 46 | 2-4:1 | 25990.85 | 46 | 2-4:1 | 5254.61 | 46 | 2-4:1 | 29.53 |
| 47 | 2-4:2 | 26428.6 | 47 | 2-4:2 | 4693.78 | 47 | 2-4:2 | 25.88 |
| 48 | 2-4:3 | 25165.79 | 48 | 2-4:3 | 4490.97 | 48 | 2-4:3 | 24.27 |
| 49 | 2-4:4 | 29413.18 | 49 | 2-4:4 | 4836.14 | 49 | 2-4:4 | 26.12 |
| 50 | 2-4:5 | 25478.91 | 50 | 2-4:5 | 4184.69 | 50 | 2-4:5 | 23.08 |
| 51 | 2-5:1 | 26476.14 | 51 | 2-5:1 | 5893.81 | 51 | 2-5:1 | 32.47 |
| 52 | 2-5:2 | 26481.86 | 52 | 2-5:2 | 4790.09 | 52 | 2-5:2 | 26.53 |
| 53 | 2-5.3 | 26520.58 | 53 | 2-5:3 | 4887.64 | 53 | 2-5:3 | 26.52 |
| 54 | 2-5:4 | 26629.56 | 54 | 2-5:4 | 4953.05 | 54 | 2-5:4 | 26.85 |
| 55 | 2-5:5 | 25985.88 | 55 | 2-5:5 | 4656.07 | 55 | 2-5:5 | 25.26 |
| 56 | 2-6:1 | 27187.69 | 56 | 2-6:1 | 6415.64 | 56 | 2-6:1 | 35.66 |
| 57 | 2-6:2 | 27032.69 | 57 | 2-6:2 | 5974.1 | 57 | 2-6:2 | 32.77 |
| 58 | 2-6:3 | 27004.19 | 58 | 2-6:3 | 5712.7 | 58 | 2-6:3 | 31.69 |
| 59 | 2-6:4 | 30952.17 | 59 | 2-6:4 | 6182.59 | 59 | 2-6:4 | 34.78 |
| 60 | 2-6:5 | 26584.3 | 60 | 2-6:5 | 5496.89 | 60 | 2-6:5 | 30.62 |
| Mean | | 24562.05 | Mean | | 4413.85 | Mean | | 23.98 |
| SD | | 2771.98 | SD | | 1246.1 | SD | | 7.35 |
| Min | | 17940.81 | Mm | | 2346.19 | Mm | | 11.51 |
| Max | | 30952.17 | Max | | 7323.1 | Max | | 41.06 |
| Range | | 13011.36 | Range | | 4976.91 | Range | | 29.55 |
| Sum | | 1473723.1 | Sum | | 264831.28 | Sum | | 1438.78 |

### EXAMPLE 3

Bipartite Probe and Single Amplification Probe Molecule for Forensic

200 ng/μl of human genomic DNA was isolated using Sambrook, J., Fritsch, E. F., and Maniatis, T., in Molecular Cloning: A Laboratory Manual. Cold Spring Harbor Laboratory Press N.Y., Vol. 1, 2, 3 (1989). 4.5 μl of 20×SSC (Saline Sodium Citrate) and 24.5 μl of human genomic DNA was added to the wells of a Whatman (Clifton, N.J.) polypropylene 384, 80 μl V-bottom wellplate and mixed. The genomic DNA was printed with a SpotBot (Telechem, Sunnyvale, Calif.) onto Superaldehyde substrates (Telechem, Sunnyvale, Calif.) in a grid pattern of five columns by six rows. The printed substrate were dried and loaded into a heating cassette. The heating cassette is composed of a beveled top plate, prefabricated spacers, a metal frame and tension clips. The substrate was lowered into the metal frame and spacers were placed on top of the substrate running lengthwise along the edge. The beveled top plate was then lowered on top of the substrate only separated by the spacers. The metal tension clips were then applied to the heating cassette, which held the cassette together securely.

The heating cassette was placed on the exterior platform of the heating block. The heating block's exterior surface is thermally controlled by different temperature fluids being perfused by external circulator baths. The contact between the heating cassette substrate and the heating block permits a highly efficient thermal transfer. The slide was then washed six times with 350 μl of deionized water. The remaining reactive aldehyde groups were removed from the slide by washing the slide six times with 350 μl of Sodium Borohydrate (0.4 g NaBH_{4, 10}mls 100% ethanol, 30mls of PBS) followed by six washes with 350 μl of deionized water.

Synthetic single stranded DNA oligonucleotide 47 mer bipartite were synthesized specific for a polymorphism in the Variable Number of Tandem Repeats (VNTR) of the human genome at the loci D1S80. The specific polymorphism identified was at the third position with a substitution of adenine for guanine. However, sequence analysis has revealed many more polymorphic sites within the D1S80 locus. D1S80 consists of a 16 base pair repeat with 29 alleles ranging in size from 369 to 800 base pairs corresponding to 14 and 41 repeats, respectively. The dry bipartites with a sequence of:

5′—GGC CGA CTC ACT GCG CGT CTT CTG TCC CGC C GAA GAC CAC CGG CAA G—3′ (SEQ ID NO: 30) was rehydrated with deionized water at a concentration of 200 μM. 2 μl of 200 μm bipartite was added to 118 μl of 0.25 NaPO₄, 4.5% SDS, 1 mMEDTA, 1×SSC, 233 Denhardt's Solution which was added to the substrate and incubated at 45° C. for 60 minutes. The substrate was washed three times with 350 μl of 2×SSC followed by two 350 μl washes in 0.2×SSC. 9 μl of CY3 Primary Detectable Amplification Molecules and 111 μl of 40% Formamide, 4×SSC, 1% SDS, 2× Denhardt's Solution, was incubated at 53° C. for 60 minutes. The substrate was washed seven times with 350 μl of 2×SSC, 0.2% SDS, followed by eleven 350 μl washes in 2×SSC and two 350μl washes in a 0.2×SSC to remove any excess dendrimer. The slides was dried and scanned. The detectable amplification molecule was detected and correlated with the presence or the amount of the said target nucleic acid sequence.

Although the present invention has been described and illustrated with respect to a preferred embodiment and a preferred use thereof, it is not to be so limited since modifications and changes can be made therein which are fully within the scope of the invention.

We claim:

1. A method for detection or quantitation of a target genetic sequence in a genetic sample an assay which comprises using:
(1) a bipartite probe consisting essentially of a target nucleic acid binding sequence capable of hybridizing said target genetic sequence and an nucleic acid binding probe sequence capable of hybridizing with a nucleic acid sequence of a primary detectable amplification molecule; and
(2) a primary detectable amplification molecule consisting essentially of a nucleic acid sequence capable of hybridizing the binding probe sequence of said bipartite probe.

2. The method of claim 1 wherein said primary amplification molecule further consists of a nucleic acid binding sequence capable of hybridizing a labeled genetic sequence.

3. The method of claim 2 wherein said labeled genetic sequence is a single stranded oligonucleotide.

4. The method of claim 2 wherein said primary detectable amplification molecule further consists of a nucleic acid sequence capable of hybridizing to a secondary detectable amplification molecule.

5. The method of claim 4 wherein said secondary detectable amplification molecule is a dendrimer.

6. The method of claim 1 wherein said primary detectable amplification molecule is a dendrimer.

7. The method of claim 1 wherein said genetic sample is genomic DNA.

8. The method of claim 2 wherein the label of said labeled genetic sequence is selected from the group consisting of enzymes, radioactive isotopes, flurogenic, chemiluminescent, electrochemical materials and members of a specific binding pair.

9. The method of claim 1 wherein said method further consists of sequentially hybridizing a second detectable amplification molecule.

10. The method of claim 9 wherein said method further consists of sequentially hybridizing a third detectable amplification molecule.

11. The method of claim 10 wherein said method further consists of sequentially hybridizing a fourth detectable amplification molecule.

12. The method of claim 1 wherein the said target nucleic acid binding sequence for a eukaryotic organism ranges from between 16 to 20 bases.

13. The method of claim 1 wherein the said target nucleic acid binding sequence for a prokaryotic organism ranges for between 8 to 13 bases.

14. The method of claim 1 wherein the said nucleic acid binding sequence is 31 bases.

15. The method of claim 1 wherein the said nucleic acid binding probe sequence of the secondary amplification molecule is 31 bases in length.

16. A method to increase signal strength in an assay for a target genetic sequence in a genetic sample comprising: hybridizing at least one detectable amplification molecule with a bipartite probe in a hybridization solution, said bipartite probe consisting essentially of a target nucleic acid binding sequence capable of hybridizing said target genetic sequence and a binding probe sequence capable of hybridizing with a nucleic acid sequence of at least one detectable amplification molecule.

17. The method of claim 16 wherein said at least one detectable amplification molecule includes at least one capture arm.

18. The method of claim 17 wherein said at least one capture arm is between 16 to 90 bases.

19. The method of claim 16 wherein said at least one detectable amplification molecule is a dendrimer.

20. A method to increase signal strength in an assay for a target genetic sequence in a genetic sample comprising: hybridizing primary and secondary detectable amplification molecules in a hybidization solution and adding a bipartite probe said bipartite probe consisting essentially of a target nucleic acid binding sequence capable of hybridizing said target genetic sequence and a binding probe sequence capable of hybridizing with a nucleic acid sequence of at least one detectable amplification molecule.

21. The method of claim 20 wherein said primary and secondary detectable amplification molecules are dendrimers.

22. The method of claim 20 wherein said detectable amplification molecules include at least one capture arm.

23. The method of claim 20 wherein said capture arms are between 16 to 90 bases.

24. A method of forensic analysis for a target nucleic acid sequence comprising:
(a) isolating human genomic DNA;
(b) immobilizing said human genomic DNA on a flat substrate;
(c) hybridizing said human genomic DNA with a bipartite probe consisting essentially of a target nucleic acid binding sequence capable of hybridizing said target genetic sequence and a binding probe sequence capable of hybridizing with a nucleic acid sequence of a primary detectable amplification molecule; and a primary detectable amplification molecule consisting essentially of a nucleic acid sequence capable of hybridizing the binding probe sequence of said bipartite probe;
(d) detecting said detectable amplification molecule; and
(e) correlating said detectable amplification molecule with said target nucleic acid sequence.

25. The method of claim 24 wherein said target nucleic acid sequence is polymorphism in the variable number of tandem repeats of the human genome.

26. The method of claim 24 wherein said target nucleic acid binding sequence capable of hybridizing said target genetic sequence comprises SEQ ID NO: 30.
